SEQUENCE LISTING (37 sequences)

SEQ ID NO: 1 - protein, 526 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 2 - protein, 8 aa, Artificial Sequence
  Amino acid seqeunce for anti-CEACAM1 antibody of  CDR1 (VH)
GFTFSNYA

SEQ ID NO: 3 - protein, 8 aa, Artificial Sequence
  Amino acid seqeunce for anti-CEACAM1 antibody of  CDR2 (VH)
ISHGGGSI

SEQ ID NO: 4 - protein, 14 aa, Artificial Sequence
  Amino acid seqeunce for anti-CEACAM1 antibody of  CDR3 (VH)
ARDPTKGYAPTFDY

SEQ ID NO: 5 - protein, 8 aa, Artificial Sequence
  Amino acid seqeunce for anti-CEACAM1 antibody of  CDR1 (VL)
SSNIGNNY

SEQ ID NO: 6 - protein, 6 aa, Artificial Sequence
  Amino acid seqeunce for anti-CEACAM1 antibody of  CDR2 (VL)
ADSRRP

SEQ ID NO: 7 - protein, 11 aa, Artificial Sequence
  Amino acid seqeunce for anti-CEACAM1 antibody of  CDR3 (VL)
GAWDLSLNGYV

SEQ ID NO: 8 - protein, 121 aa, Artificial Sequence
  Amino acid seqeunce for anti-CEACAM1 antibody of variable heavy region
EVQLLESGGGLVQPGGSLRLSCAASGFTFSNYAMSWVRQAPGKGLEWVSVISHGGGSIYYADSVKGRLTISRDNSKNTLYLQMNSLRAEDTAVYYCARDPTKGYAPTFDYWGQGTLVTVSS

SEQ ID NO: 9 - protein, 111 aa, Artificial Sequence
  Amino acid seqeunce for anti-CEACAM1 antibody of  variable light region
QSVLTQPPSASGTPGQRVTISCSGSSSNIGNNYVSWYQQLPGTAPKLLIYADSRRPSGVPDRFSGSKSGTSASLAISGLRSEDEADYYCGAWDLSLNGYVFGGGTKLTVLG

SEQ ID NO: 10 - DNA, 363 bp, Artificial Sequence
  Nucleotide seqeunce for anti-CEACAM1 antibody of variable heavy region
gaggtgcagctgttggagtctgggggaggcttggtacagcctggggggtccctgagactctcctgtgcagcctctggattcacctttagcaattatgctatgagctgggtccgccaggctccagggaaggggctggagtgggtctcagtgatctctcatggtggtggtagtatatattacgctgattctgtaaaaggtcggctcaccatctccagagacaattccaagaacacgctgtatctgcaaatgaacagcctgagagccgaggacacggccgtgtattactgtgcgagagatcctactaaggggtatgctcctactttcgactactggggccagggtacactggtcaccgtgagctca

SEQ ID NO: 11 - DNA, 333 bp, Artificial Sequence
  Nucleotide seqeunce for anti-CEACAM1 antibody of  variable light region
cagtctgtgctgactcagccaccctcagcgtctgggacccccgggcagagggtcaccatctcttgtagtggctcttcatccaatattggcaataattatgtctcctggtaccagcagctcccaggaacggcccccaaactcctcatctatgctgatagtaggcggccaagcggggtccctgaccgattctctggctccaagtctggcacctcagcctccctggccatcagtgggctccggtccgaggatgaggctgattattactgtggtgcttgggatctgagcctgaatggttatgtcttcggcggaggcaccaagctgaccgtcctaggt

SEQ ID NO: 12 - DNA, 1401 bp, Artificial Sequence
  Nucleotide seqeunce for anti-CEACAM1 antibody of heavy chain(IgG4)
atgtacttgggactgaactatgtattcatagtttttctcttaaatggtgtccagagtgaggtgcagctgttggagtctgggggaggcttggtacagcctggggggtccctgagactctcctgtgcagcctctggattcacctttagcaattatgctatgagctgggtccgccaggctccagggaaggggctggagtgggtctcagtgatctctcatggtggtggtagtatatattacgctgattctgtaaaaggtcggctcaccatctccagagacaattccaagaacacgctgtatctgcaaatgaacagcctgagagccgaggacacggccgtgtattactgtgcgagagatcctactaaggggtatgctcctactttcgactactggggccagggtacactggtcaccgtgagctcagccagcaccaagggaccctccgtgttcccactggcaccctgctcccggagcaccagcgagagcaccgccgccctgggatgtctggtgaaagactacttccctgaaccagtcaccgtgtcctggaactctggcgcactgacttccggagtccacacctttcccgccgtgctccagagcagcggactgtactctctgtcaagcgtggtcacagtcccctcctctagtctgggaacaaagacttatacctgcaacgtggatcataaaccttccaatactaaggtggacaaaagagtggagtctaagtacggccctccttgcccaccatgtccagcacctgaatttctgggcggaccctccgtgttcctgtttcctccaaagcctaaagatacactgatgatctcccgcacccctgaggtcacatgcgtggtcgtggacgtgagccaggaggacccagaagtccagttcaactggtatgtggacggcgtcgaagtgcacaatgctaagaccaaacctagggaggaacagtttaactctacttacagagtcgtgagtgtcctgaccgtgctgcatcaggattggctgaacggcaaggagtataagtgcaaagtgagcaataagggactgccatcaagcatcgagaaaaccattagcaaggcaaaaggccagcctcgcgaaccacaggtgtacacactgccccctagtcaggaggaaatgaccaagaaccaggtcagcctgacatgtctggtgaaagggttctatccatcagatattgctgtggagtgggaaagcaatggtcagcccgagaacaattacaagactaccccacccgtgctggacagtgatgggtcattctttctgtattctcgactgaccgtggacaaaagtcggtggcaggagggtaatgtcttttcatgcagcgtgatgcacgaggcactgcacaaccattacactcagaagtcactgtcactgtcactgggaaag

SEQ ID NO: 13 - protein, 467 aa, Artificial Sequence
  Amino acid seqeunce for anti-CEACAM1 antibody of heavy chain(IgG4)
MYLGLNYVFIVFLLNGVQSEVQLLESGGGLVQPGGSLRLSCAASGFTFSNYAMSWVRQAPGKGLEWVSVISHGGGSIYYADSVKGRLTISRDNSKNTLYLQMNSLRAEDTAVYYCARDPTKGYAPTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - DNA, 1059 bp, Artificial Sequence
  Nucleotide seqeunce for CH1-hinge-CH2- CH3(hIgG1)
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaacccaaatcttgtgacaaaactcacacatgcccaccgtgcccagcacctgaacttccttggggggaccgtcaattcttcctcttccccccaaaacccaaggaaccccctcttgatgaactccttgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 15 - protein, 352 aa, Artificial Sequence
  Amino acid seqeunce for CH1-hinge-CH2- CH3(hIgG1)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELPWGDRQFFLFPPKPKEPPLDELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 66 bp, Artificial Sequence
  Forward primer for VH-NotI-L
gcggccgccatgtacttgggactgaactatgtattcatagtttttctcttaaatggtgtccagagt

SEQ ID NO: 17 - DNA, 34 bp, Artificial Sequence
  Reverse primer for VH-ApaI-CH1
atgggcccttggtggaggctgaggagacggtgac

SEQ ID NO: 18 - DNA, 18 bp, Artificial Sequence
  Forward primer for CH1-Fc_ApaI
gcctccaccaagggccca

SEQ ID NO: 19 - DNA, 30 bp, Artificial Sequence
  Reverse primer for IgG1_Fc_HindIII
  (1)..(4): n is a or g or c or t
nnnnggatccaagcttactatttacccgga

SEQ ID NO: 20 - DNA, 19 bp, Artificial Sequence
  Forward primer for IgG1_HC_NotI
gcggccgccatgtacttgg

SEQ ID NO: 21 - DNA, 87 bp, Artificial Sequence
  Forward primer for NotI-SS-CEACAM1
gcggccgccatgtacttgggactgaactatgtattcatagtttttctcttaaatggtgtccagagtcagctcactactgaatccatg

SEQ ID NO: 22 - DNA, 45 bp, Artificial Sequence
  Reverse primer for CEACAM1-Hinge
gcaaggagggccgtacttagactccccaggtgagaggccattttc

SEQ ID NO: 23 - DNA, 1182 bp, Artificial Sequence
  Nucleotide seqeunce for human CEACAM1 extracellular domain
cagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagctaagtccagtagtagcaaagccccaaatcaaagccagcaagaccacagtcacaggagataaggactctgtgaacctgacctgctccacaaatgacactggaatctccatccgttggttcttcaaaaaccagagtctcccgtcctcggagaggatgaagctgtcccagggcaacaccaccctcagcataaaccctgtcaagagggaggatgctgggacgtattggtgtgaggtcttcaacccaatcagtaagaaccaaagcgaccccatcatgctgaacgtaaactataatgctctaccacaagaaaatggcctctcacctggg

SEQ ID NO: 24 - DNA, 24 bp, Artificial Sequence
  Forward primer for hIgG4-Hinge
gagtctaagtacggccctccttgc

SEQ ID NO: 25 - DNA, 34 bp, Artificial Sequence
  Reverse primer for hIgG4-CH3-stop HindIII
ggatccaagcttactactttcccagtgacagtga

SEQ ID NO: 26 - DNA, 699 bp, Artificial Sequence
  Nucleotide seqeunce for Human Ig gamma-4 chain C region (codon optimization in Ab team)
gagtctaagtacggccctccttgcccaccatgtccagcacctgaatttctgggcggaccctccgtgttcctgtttcctccaaagcctaaagatacactgatgatctcccgcacccctgaggtcacatgcgtggtcgtggacgtgagccaggaggacccagaagtccagttcaactggtatgtggacggcgtcgaagtgcacaatgctaagaccaaacctagggaggaacagtttaactctacttacagagtcgtgagtgtcctgaccgtgctgcatcaggattggctgaacggcaaggagtataagtgcaaagtgagcaataagggactgccatcaagcatcgagaaaaccattagcaaggcaaaaggccagcctcgcgaaccacaggtgtacacactgccccctagtcaggaggaaatgaccaagaaccaggtcagcctgacatgtctggtgaaagggttctatccatcagatattgctgtggagtgggaaagcaatggtcagcccgagaacaattacaagactaccccacccgtgctggacagtgatgggtcattctttctgtattctcgactgaccgtggacaaaagtcggtggcaggagggtaatgtcttttcatgcagcgtgatgcacgaggcactgcacaaccattacactcagaagtcactgtcactgtcactgggaaagtagtaaaagctt

SEQ ID NO: 27 - DNA, 33 bp, Artificial Sequence
  Forward primer for SS_hCEACAM6
aatggtgtccagagtaagctcactattgaatcc

SEQ ID NO: 28 - DNA, 36 bp, Artificial Sequence
  Reverse primer for hCEACAM6_R_hinge(HIgG1)
gtcacaagatttgggctctccagagactgtgatcat

SEQ ID NO: 29 - DNA, 18 bp, Artificial Sequence
  Forward primer for hinge(HIgG1)
gagcccaaatcttgtgac

SEQ ID NO: 30 - DNA, 36 bp, Artificial Sequence
  Reverse primer for CH3(HIgG1)_HindIII
ttggatccaagcttactatttacccggagacaggga

SEQ ID NO: 31 - DNA, 72 bp, Artificial Sequence
  Forward primer for NotI-SS
  (1)..(6): n is a or g or c or t
nnnnnngcggccgccatgtacttgggactgaactatgtattcatagtttttctcttaaatggtgtccagagt

SEQ ID NO: 32 - DNA, 1556 bp, Artificial Sequence
  Nucleotide seqeunce for hCEACAM6(ECD)-Fc
aagctcactattgaatccacgccattcaatgtcgcagaggggaaggaggttcttctactcgcccacaacctgccccagaatcgtattggttacagctggtacaaaggcgaaagagtggatggcaacagtctaattgtaggatatgtaataggaactcaacaagctaccccagggcccgcatacagtggtcgagagacaatataccccaatgcatccctgctgatccagaacgtcacccagaatgacacaggattctataccctacaagtcataaagtcagatcttgtgaatgaagaagcaaccggacagttccatgtatacccggagctgcccaagccctccatctccagcaacaactccaccccgtggaggacaaggatgctgtggccttcacctgtgaacctgaggttcagaacacaacctacctgtggtgggtaaatggtcagagcctcccggtcagtcccaggctgcagctgtccaatggcaacatgaccctcactctactcagcgtcaaaaggaacgatgcaggatcctatgaatgtgaaatacagaacccagcgagtgccaaccgcagtgacccagtcaccctgaatgtcctctatggcccagatgtccccaccatttccccctcaaaggccaattaccgtccaggggaaaatctgaacctctcctgccacgcagcctctaacccacctgcacagtactcttggtttatcaatgggacgttccagcaatccacacaagagctctttatccccaacatcactgtgaataatagcggatcctatatgtgccaagcccataactcagccactggcctcaataggaccacagtcacgatgatcacagtctctggagagcccaaatcttgtgacaaaactcacacatgcccaccgtgcccagcacctgaactccttgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatag

SEQ ID NO: 33 - DNA, 1419 bp, Artificial Sequence
  Nucleotide seqeunce for anti-CEACAM1 antibody HC_IgG1 type
gaggtgcagctgttggagtctgggggaggcttggtacagcctggggggtccctgagactctcctgtgcagcctctggattcacctttagcaattatgctatgagctgggtccgccaggctccagggaaggggctggagtgggtctcagtgatctctcatggtggtggtagtatatattacgctgattctgtaaaaggtcggctcaccatctccagagacaattccaagaacacgctgtatctgcaaatgaacagcctgagagccgaggacacggccgtgtattactgtgcgagagatcctactaaggggtatgctcctactttcgactactggggccagggtacactggtcaccgtgagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaacccaaatcttgtgacaaaactcacacatgcccaccgtgcccagcacctgaacttccttggggggaccgtcaattcttcctcttccccccaaaacccaaggaaccccctcttgatgaactccttgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 34 - DNA, 1929 bp, Artificial Sequence
  Nucleotide seqeunce for pCIW_HC_CCM1-FcIgG4
atgtacttgggactgaactatgtattcatagtttttctcttaaatggtgtccagagtcagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagctaagtccagtagtagcaaagccccaaatcaaagccagcaagaccacagtcacaggagataaggactctgtgaacctgacctgctccacaaatgacactggaatctccatccgttggttcttcaaaaaccagagtctcccgtcctcggagaggatgaagctgtcccagggcaacaccaccctcagcataaaccctgtcaagagggaggatgctgggacgtattggtgtgaggtcttcaacccaatcagtaagaaccaaagcgaccccatcatgctgaacgtaaactataatgctctaccacaagaaaatggcctctcacctggggagtctaagtacggccctccttgcccaccatgtccagcacctgaatttctgggcggaccctccgtgttcctgtttcctccaaagcctaaagatacactgatgatctcccgcacccctgaggtcacatgcgtggtcgtggacgtgagccaggaggacccagaagtccagttcaactggtatgtggacggcgtcgaagtgcacaatgctaagaccaaacctagggaggaacagtttaactctacttacagagtcgtgagtgtcctgaccgtgctgcatcaggattggctgaacggcaaggagtataagtgcaaagtgagcaataagggactgccatcaagcatcgagaaaaccattagcaaggcaaaaggccagcctcgcgaaccacaggtgtacacactgccccctagtcaggaggaaatgaccaagaaccaggtcagcctgacatgtctggtgaaagggttctatccatcagatattgctgtggagtgggaaagcaatggtcagcccgagaacaattacaagactaccccacccgtgctggacagtgatgggtcattctttctgtattctcgactgaccgtggacaaaagtcggtggcaggagggtaatgtcttttcatgcagcgtgatgcacgaggcactgcacaaccattacactcagaagtcactgtcactgtcactgggaaagtag

SEQ ID NO: 35 - DNA, 28 bp, Artificial Sequence
  Forward primer for CEACAM1_HindIII
gacaagcttatggggcacctctcagccc

SEQ ID NO: 36 - DNA, 33 bp, Artificial Sequence
  Reverse primer for CEACAM1_SalI
gacgtcgacgtctgcttttttacttctgaataa

SEQ ID NO: 37 - DNA, 1562 bp, Artificial Sequence
  Nucleotide seqeunce for human CEACAM1
atggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagctaagtccagtagtagcaaagccccaaatcaaagccagcaagaccacagtcacaggagataaggactctgtgaacctgacctgctccacaaatgacactggaatctccatccgttggttcttcaaaaaccagagtctcccgtcctcggagaggatgaagctgtcccagggcaacaccaccctcagcataaaccctgtcaagagggaggatgctgggacgtattggtgtgaggtcttcaacccaatcagtaagaaccaaagcgaccccatcatgctgaacgtaaactataatgctctaccacaagaaaatggcctctcacctggggccattgctggcattgtgattggagtagtggccctggttgctctgatagcagtagccctggcatgttttctgcatttcgggaagaccggcagggcaagcgaccagcgtgatctcacagagcacaaaccctcagtctccaaccacactcaggaccactccaatgacccacctaacaagatgaatgaagttacttattctaccctgaactttgaagcccagcaacccacacaaccaacttcagcctccccatccctaacagccacagaaataatttataa

(19) United States
(12) Patent Application Publication
(10) Pub. No.: US 2022/0289819 A1
(43) Pub. Date: Sep. 15, 2022

(54) STERIC EPITOPE OF CEACAM1, AND ANTI-CEACAM1 ANTIBODY, OR FRAGMENT THEREOF, THAT SPECIFICALLY BINDS TO SAME

(71) Applicants: GREEN CROSS CORPORATION, Yongin, Gyeonggi-do (KR); MOGAM INSTITUTE FOR BIOMEDICAL RESEARCH, Yongin-si, Gyeonggi-do (KR)

(72) Inventors: Mi-Young OH, Yongin-si (KR); Jae-Chul LEE, Yongin-si (KR); Hye Young PARK, Yongin-si (KR); Hye In YUM, Yongin-si (KR); Dong-Sik KIM, Yongin-si (KR)

(73) Assignees: GREEN CROSS CORPORATION, Yongin, Gyeonggi-do (KR); MOGAM INSTITUTE FOR BIOMEDICAL RESEARCH, Yongin-si, Gyeonggi-do (KR)

(21) Appl. No.: 17/770,500

(22) PCT Filed: Nov. 7, 2019
(86) PCT No.: PCT/KR2019/015080

§ 371 Date: Apr. 20, 2022

(51) Int. Cl. C07K 14/705; C07K 16/28; A61P 35/00

(52) U.S. Cl. CPC C07K 14/70503; C07K 16/2803; A61P 35/00; C07K 2317/34; C07K 2317/92; C07K 2317/55; C07K 2317/76

(57) ABSTRACT

A steric epitope of CEACAM1 is disclosed. An anti-CEACAM1 antibody or a fragment thereof, which specifically binds to CEACAM1 is disclosed. A steric epitope of CEACAM1 includes all amino acids in critical positions for specific binding to an anti-CEACAM1 antibody and maintains an appropriate three-dimensional structure, and thus can high affinity for an anti-CEACAM1 antibody. In addition, an antibody, or a fragment thereof, that specifically binds to a steric epitope can effectively suppress CEACAM1-CEACAM1 interaction and CEACAM1-CEACAM6 interaction.

### TECHNICAL FIELD

The present invention relates to a conformational epitope of CEACAM1 and an anti-CEACAM1 antibody or a fragment thereof that specifically binds thereto.

### BACKGROUND ART

Carcinoembryonic antigen-related cell adhesion molecule 1 (CEACAM1) is one of the transmembrane glycoproteins belonging to the group of carcinoembryonic antigens. CEACAM1 is mainly expressed in activated T cells and natural killer cells, and also shows high expression in cancer cells.

CEACAM1 plays a role in regulating innate and adaptive immune responses. In this regard, in the case of cancer cells where CEACAM1 is overexpressed, CEACAM1-CEACAM1 interacts with T cells where CEACAM1 is expressed. Once the CEACAM1-CEACAM1 interaction occurs, Src homology region 2 domain-containing phosphatase-1 (SHP1) binds to an immunoreceptor tyrosine-based inhibition motif (ITIM) portion of CEACAM1, which is phosphorylated by the lymphocyte-specific protein tyrosine kinase (LCK) and is bound to the CD4 terminus of the T cell receptors (TCRs) of the T cells. Moreover, there was a research report that when the CD3ζ end is dephosphorylated by the SHP1 protein, the RAS-MAPK signaling mechanism is not activated and thus T cells are not activated, thereby allowing cancer cells to evade the immune response (Scott D. Gray-Owen & Richard S. Blumberg, Nature Reviews Immunology, volume 6, pages 433-446, 2006).

In addition, CEACAM1 has not only a homophilic interaction, but also a heterophilic interaction with CEACAM5 or CEACAM6. Among them, CEACAM6 is expressed in various carcinomas (e.g., breast tumors, pancreatic tumors, ovarian adenocarcinomas, lung adenocarcinoma, etc.) (Blumenthal et al. BMC Cancer, 2007 Jan. 3; 7:2). There was a research report that CEACAM1 expressed in activated T cells inhibits TCR signals through its binding to CEACAM6, and this prevents T cells from being activated by a mechanism as in the CEACAM 1-CEACAM1 interaction, thereby allowing CEACAM6-expressing cancer cells to evade the immune response (Witzens-Harig et al., Blood 2013 May 30:121(22):4493-503).

Accordingly, the inhibition of the CEACAM1-CEACAM1 interaction in cancer cells has emerged as a promising anticancer therapy, there is a need for the identification of the exact conformational epitope of CEACAM1 so as to produce and confirm an antibody against CEACAM1 that can effectively inhibit the CEACAM 1-CEACAM1 interaction.

### DISCLOSURE OF INVENTION

### Technical Problem

As such, in order to confirm the conformational epitope of CEACAM1, the present inventors have crystallized the structure of the complex in which an anti-CEACAM1 antibody and CEACAM1 are conjugated through X-ray diffraction (XRD), and have confirmed that the antibody specifically binding to the conformational epitope inhibits the CEACAM1-CEACAM1 interaction and the CEACAM1-CEACAM6 interaction, thereby completing the present disclosure.

### Solution to Problem

To achieve the above objects, an aspect of the present disclosure provides a conformational epitope consisting of 4 to 69 amino acids of a sequence of amino acids at positions 35 to 141 of CEACAM1, wherein the conformational epitope comprises any one amino acid selected from the group consisting of the amino acids at positions 63, 64, 66, 68, 75, 76, 78, 83, 86, 90, 123, 125, 129, and 131, and a combination thereof.

Another aspect of the present disclosure provides an anti-CEACAM1 antibody or a fragment thereof, which specifically binds to a conformational epitope consisting of 4 to 69 amino acids of a sequence of amino acids at positions 35 to 141 of CEACAM1, wherein the conformational epitope comprises any one amino acid selected from the group consisting of the amino acids at positions 63, 64, 66, 68, 75, 76, 78, 83, 86, 90, 123, 125, 129, and 131, and a combination thereof.

### Advantageous Effects of Invention

The conformational epitope of CEACAM1 of the present disclosure shows high affinity for an anti-CEACAM1 antibody by maintaining an appropriate three-dimensional structure while including all of the amino acids at positions important for specific binding to the anti-CEACAM1 antibody. In addition, the antibody or a fragment thereof that specifically binds to a conformational epitope according to the present disclosure can effectively inhibit the CEACAM1-CEACAM1 interaction and the CEACAM1-CEACAM6 interaction.

### BRIEF DESCRIPTION OF DRAWINGS

FIG. 1 is a drawing showing the crystal structures of a complex of a Fab of an anti-CEACAM1 antibody according to an embodiment and the N-domain of CEACAM1 by X-ray diffraction (XRD) analysis.

FIG. 2 is a drawing showing the structure of a complex of a variable region of an anti-CEACAM1 antibody according to an embodiment and the N-domain of CEACAM1 (left); and the structure of a dimeric binding structure between N-domains of CEACAM1 (right).

FIG. 3 is a drawing showing the amino acids in the portion, where a Fab of an anti-CEACAM1 antibody according to an embodiment and the N-domain of CEACAM1 are bound.

FIG. 4 is a drawing showing the amino acids that form a dimeric binding between N-domains of CEACAM1.

FIGS. 5A to 5D are drawings showing amino acids in which the intermolecular distance between a Fab of an anti-CEACAM1 antibody and the N-domain of CEACAM1 is within 4.5 Å.

FIG. 6 is a drawing showing the sequence information of the amino acids of the hCEACAM1 extracellular domain and the DNA encoding the same.

FIG. 7 is a drawing showing the sequence information of the amino acids in the hIgG4 Fc region and DNA encoding same.

FIG. 8 is a drawing confirming the homophilic interaction between CEACAM1-Fc and CEACAM1-Fc according to the concentration of CEACAM1-Fc.

FIG. 9 is a drawing confirming the inhibitory effect on the homophilic interaction between CEACAM1-Fe and CEACAM1-Fc according to the concentration of an anti-CEACAM1 antibody according to an embodiment.

FIG. 10 is a drawing showing the sequence information of DNA encoding hCEACAM6(ECD)-Fc.

FIG. 11 is a drawing confirming the heterophilic interaction between CEACAM1-Fc and CEACAM6-Fc according to the concentration of CEACAM1-Fc.

FIG. 12 is a drawing confirming the inhibitory effect on the heteroaffinity interaction between CEACAM1-Fc and CEACAM6-Fc according to the concentration of an anti-CEACAM1 antibody according to an embodiment.

FIG. 13 is a drawing confirming the effect of increasing ZAP70 phosphorylation of an anti-CEACAM1 antibody according to an embodiment in CEACAM1-overexpressing Jurkat cells.

FIG. 14 is a drawing confirming the effect of increasing NFAT expression of an anti-CEACAM1 antibody according to an embodiment in Jurkat cells, which overexpress NFAT and CEACAM1.

FIG. 15 is a drawing confirming the effect of increasing IL-2 expression of the anti-CEACAM1 antibody according to an embodiment in CEACAM1-overexpressing Jurkat cells.

### BEST MODE FOR CARRYING OUT THE INVENTION

Hereinafter, the present disclosure will be described in detail.

An aspect of the present disclosure provides a conformational epitope consisting of 4 to 69 amino acids of a sequence of amino acids at positions 35 to 141 of CEACAM1, wherein the conformational epitope comprises any one amino acid selected from the group consisting of the amino acids at positions 63, 64, 66, 68, 75, 76, 78, 83, 86, 90, 123, 125, 129, and 131, and a combination thereof.

The carcinoembryonic antigen-related cell adhesion molecule 1 (CEACAM1) is one of the transmembrane glycoproteins belonging to the group of carcinoembryonic antigens. The CEACAM1 is mainly expressed in activated T cells and natural killer cells, and also shows high expression in cancer cells. The amino acid sequence at positions 35 to 141 of CEACAM1 corresponds to the N-domain of CEACAM1, and is known to be involved in the CEACAM1-CEACAM1 interaction or the CEACAM1-CEACAM6 interaction. The CEACAM1 may be human CEACAM1, and the amino acid sequence of human CEACAM1 may be an amino acid sequence of SEQ ID NO: 1.

The 63rd amino acid is phenylalanine (Phe), the 64th amino acid is glycine (Gly), the 66th amino acid is serine (Ser), the 68th amino acid is tyrosine (Tyr), the 75th amino acid is glycine (Gly), the 76th amino acid is asparagine (Asn), the 78th amino acid is glutamine (Gln), the 83rd amino acid is alanine (Ala), the 86th amino acid is threonine (Thr), the 90th amino acid is threonine (Thr), the 123rd amino acid is glutamine (Gin), the 125th amino acid is isoleucine (Ile), the 129th amino acid is leucine (Leu), and the 131st amino acid is asparagine (Asn).

The conformational epitope may include adjacent amino acids or non-adjacent amino acids due to three-dimensional folding of the protein. Specifically, the conformational epitope may include at least 4, 5, 9, or 10 amino acids in a conformational structure of an independent space.

Specifically, the conformational epitope may comprise amino acids at positions 63, 64, 66, and 68 of a sequence of amino acids at positions 35 to 141 of CEACAM1. In addition, the conformational epitope may comprise amino acids at positions 75, 76, 78, 83, 86, and 90 of a amino acid sequence at positions 35 to 141 of CEACAM1. In addition, the conformational epitope may comprise amino acids at positions 123, 125, 129, and 131 of a amino acid sequence at positions 35 to 141 of CEACAM1.

Furthermore, the conformational epitope may comprise amino acids at positions 63, 66, 68, 78, 123, 125, 129, and 131 of a sequence of amino acids at positions 35 to 141 of CEACAM1. In addition, the conformational epitope may comprise amino acids at positions 64, 75, 76, 83, 86, and 90 of a sequence of amino acids at positions 35 to 141 of CEACAM1. Preferably, the conformational epitope, being composed of 4 to 69 amino acids of a sequence of amino acids at positions 35 to 141 of CEACAM1, may comprise amino acids at positions 63, 64, 66, 68, 75, 76, 78, 83, 86, 90, 123, 125, 129, and 131.

In the present invention, in order to identify a novel conformational epitope of CEACAM1, the structure of a complex in which CEACAM1 and an anti-CEACAM1 antibody according to an embodiment are bound was crystallized at a resolution of 1.8 Å (FIGS. 1 to 5D). As a result, a novel epitope of CEACAM1 and a paratope of anti-CEACAM1 according to an embodiment were confirmed, and are shown in Table 1 below.

TABLE 1

| | Anti-CEACAM1 Antibody | | Type of |
|---|---|---|---|
| CEACAM1 | VH | VL | Interaction |
| Phe63 | Thr101 | | Van der Waals |
| Phe63 | Tyr104 | | Hydrophobic |
| Phe63 | Ala105 | | Van der Waals |
| Phe63 | | Tyr33 | Van der Waals |
| Phe63 | | Tyr50 | Hydrophobic |
| Phe63 | | Ala51 | Van der Waals |
| Gly64 | Tyr104 | | Van der Waals |
| Ser66 | Tyr104 | | H-bonding |
| Tyr68 | Asn31 | | H-bonding |
| Gly75 | Asn31 | | Hydrophobic |
| Asn76 | Asn74 | | Ionic interaction |
| Gln78 | Lys102 | | Ionic interaction |
| Gln78 | Tyr104 | | Ionic interaction |
| Ala83 | Tyr104 | | Hydrophobic |
| Thr86 | | Leu94 | Hydrophobic |
| Thr90 | Gly54 | | Hydrophobic |
| Thr90 | Gly56 | | Hydrophobic |
| Gln123 | Asn31 | | Ionic interaction |
| Ile125 | Lys102 | | Van der Waals |
| Ile125 | Tyr104 | | Hydrophobic |
| Leu129 | | Tyr50 | Van der Waals |
| Leu129 | Pro100 | | Van der Waals |
| Leu129 | Thr101 | | Van der Waals |
| Asn131 | Tyr32 | | Ionic interaction |

As shown in Table 1, it was confirmed that the anti-CEACAM1 antibody binds to the amino acids at positions 63, 64, 66, 68, 75, 76, 78, 83, 86, 90, 123, 125, 129, and 131 of the sequence of amino acids at positions 35 to 141 of CEACAM1.

Another aspect of the present disclosure provides an anti-CEACAM1 antibody or a fragment thereof, which specifically binds to a conformational epitope consisting of 4 to 69 amino acids of a sequence of amino acids at positions 35 to 141 of CEACAM1, wherein the conformational epitope comprises any one amino acid selected from the group consisting of the amino acids at positions 63, 64, 66, 68, 75, 76, 78, 83, 86, 90, 123, 125, 129, and 131, and a combination thereof.

The conformational epitope is the same as described above.

Specifically, the antibody or fragment thereof may specifically bind to a conformational epitope, which comprises amino acids at positions 63, 64, 66, and 68 of a sequence of amino acids at positions 35 to 141 of CEACAM1. In addition, the antibody or fragment thereof may specifically bind to a conformational epitope, which comprises amino acids at positions 75, 76, 78, 83, 86, and 90 of a amino acid sequence at positions 35 to 141 of CEACAM1. Furthermore, the antibody or fragment thereof may specifically bind to a conformational epitope, which comprises amino acids at positions 123, 125, 129, and 131 of a sequence of amino acids at positions 35 to 141 of CEACAM1.

Furthermore, the antibody or fragment thereof may specifically bind to a conformational epitope, which comprises amino acids at positions 63, 66, 68, 78, 123, 125, 129, and 131 of a sequence of amino acids at positions 35 to 141 of CEACAM1. In addition, the antibody or fragment thereof may specifically bind to a conformational epitope, which comprises amino acids at positions 64, 75, 76, 83, 86, and 90 of a sequence of amino acids at positions 35 to 141 of CEACAM1. Preferably, the antibody or fragment thereof, being composed of 4 to 69 amino acids of the sequence of amino acids at positions 35 to 141 of CEACAM1, may specifically bind to a conformational epitope, which comprises amino acids at positions 63, 64, 66, 68, 75, 76, 78, 83, 86, 90, 123, 125, 129, and 131.

The antibody or fragment thereof may bind to CEACAM1 within an intermolecular distance of 4.5 Å. The antibody or fragment thereof may have a Van der Waals bond, a hydrophobic bond, or an electrostatic bond with CEACAM1.

The antibody or fragment thereof may have a binding affinity for CEACAM1 of less than 1×10⁻⁸ KD (M). Specifically, the antibody or fragment thereof may have a binding affinity for CEACAM1 of less than 9 ×10⁻⁹, 8×10⁻⁹, 7×10⁻⁹, 6×10⁻⁹, 5×10⁻⁹, or 4×^{10 -9} KD (M). In one embodiment of the present disclosure, the antibody of the above embodiment, the binding affinity for CEACAM1 was measured to be 3.36×^{10 -9} KD (M).

The antibody or fragment thereof may include a heavy chain CDR1 including an amino acid sequence represented by SEQ ID NO: 2, a heavy chain CDR2 including an amino acid sequence represented by SEQ ID NO: 3, a heavy chain CDR3 including an amino acid sequence represented by SEQ ID NO: 4, a light chain CDR1 including an amino acid sequence represented by SEQ ID NO: 5, a light chain CDR2 including an amino acid sequence represented by SEQ ID NO: 6, and a light chain CDR3 including an amino acid sequence represented by SEQ ID NO: 7.

The antibody or fragment thereof may include a heavy chain variable region including an amino acid sequence represented by SEQ ID NO: 8; and a light chain variable region including an amino acid sequence represented by SEQ ID NO: 9.

The fragment of the antibody may be any one selected from the group consisting of Fab, scFv, F(ab′)₂, and Fv. An antibody fragment refers to antigen-binding domains excluding the fragment crystallizable region (the Fc region), which has an effector function that transmits an antigen-binding stimulus to cells, complements, etc., and it may include a third-generation antibody fragment (e.g., a single domain antibody, a minibody, etc.).

Still another aspect of the present invention provides an anticancer agent which includes the antibody or fragment thereof as an active ingredient.

An anticancer agent, which includes the antibody or a fragment thereof as an active ingredient, may be used to treat cancer or tumor where CEACAM1 is overexpressed. Specifically, when the T cell receptors (TCRs) of cytotoxic T cells, which play a role in removing cancer cells, recognize the antigenic determinant of cancer or tumor cells, the lymphocyte-specific protein tyrosine kinase (LCK) protein bound to the end of cluster of differentiation 4 (CD4) (which is one of the components of the TCR) phosphorylates a cluster of differentiation 3ζ(CD3ζ) (which is another component of TCR). When the zeta-chain-associated protein kinase 70 (ZAP70) protein binds to the phosphorylated CD3ζ portion, the end of the ZAP70 protein is phosphorylated by the LCK protein, and the Ras-MAP kinase signal transduction is activated, thereby activating the T cells.

However, in the case of cancer cells or tumor cells where CEACAM1 is overexpressed, the Src homology region 2 domain-containing phosphatase-1 (SHP 1) protein binds to the immunoreceptor tyrosine-based inhibition motif (ITIM) portion of the CEACAM1, which is phosphorylated by the LCK protein bound to the CD4 end of the TCR due to a CEACAM1-CEACAM1 interaction. In addition, the CD3ζ end is dephosphorylated by the SHP1 protein, and as a result, the RAS-MAPK signaling mechanism cannot be activated, and thus T cells are not activated.

Therefore, the antibody or fragment thereof bind to the CEACAM1 expressed in cytotoxic T cells, natural killer cells, and cancer cells, and block the CEACAM1-CEACAM1 interaction in advance, and thus can be used as an anticancer agent.

In addition, as used herein, the term “anti-cancer” includes “prevention” and “treatment”, in which “prevention” refers to all actions that inhibit the proliferation of cancer or delay the progression of cancer by the administration of the anticancer agent, and “treatment” refers to all actions that improve or beneficially change the symptoms of cancer by the administration of the antibody of the present disclosure.

In addition, as used herein, the term “cancer” may be characterized as being selected from the group consisting of pancreatic cancer, melanoma, lung cancer, and myeloma, but is not particularly limited thereto as long as it has CEACAM1 as a receptor, and the immune checkpoint pathway is abnormally operated, and may include solid cancer and hematologic cancer.

Still another aspect of the present invention provides a polynucleotide which encodes the antibody or fragment thereof. Specifically, the polynucleotide may include a nucleotide sequence represented by SEQ ID NO: 10 and/or 11.

The polynucleotide may be modified by substitution, deletion, insertion, or a combination of one or more bases. When the nucleotide sequence is prepared by chemically synthesizing the nucleotide sequence, a synthetic method well known in the art (e.g., a method described in Engels and Uhlmann, Advances in biochemical engineering/biotechnology, 37:73-127, 1988) may be used, and may include triesterphosphite, phosphoramidite, and H-phosphate methods, PCR and other autoprimer methods, an oligonucleotide synthesis method on a solid support, etc.

In addition, still another aspect of the present invention provides an expression vector including the polynucleotide. The expression vector may be plasmid DNA, phage DNA, etc., and may be commercially developed plasmids (pUC18, pBAD, pIDTSAMRT-AMP, etc.), E. coli-derived plasmids (pYG601BR322, pBR325, pUC118, pUC119, etc.), Bacillus subtilis-derived plasmids (pUB110, pTP5, etc.), yeast-derived plasmids (YEp13, YEp24, YCp50, etc.), phage DNA (Charon4A, Charon21A, EMBL3, EMBL4, λgt10, λgt11, λZAP, etc.), animal virus vectors (retrovirus, adenovirus, vaccinia virus, etc.), and insect virus vectors (baculovirus, etc.). Since the expression vector shows different expression levels and modifications of proteins depending on the host cell, it is preferable to select and use the most suitable host cell for the purpose.

Still another aspect of the present invention provides a transformed cell into which the expression vector is introduced. The host cell of the transformed cell may include the cells of mammalian, plant, insect, bacterial, or cellular origin, but is not limited thereto. As the mammalian cells, CHO cells, F2N cells, CSO cells, BHK cells, Bowes melanoma cells, HeLa cells, 911 cells, AT1080 cells, A549 cells, HEK 293 cells HEK293T cells, etc., but are not limited thereto, and any cell, which is known to those skilled in the art to be used as a mammalian host cell, can be used.

In addition, when introducing an expression vector into a host cell, methods such as a CaCl₂ precipitation method, the Hanahan method which has improved efficiency by using a reducing material called dimethyl sulfoxide (DMSO) in the CaCl₂ precipitation method, electroporation, a calcium phosphate precipitation method, a protoplasm fusion method, an agitation method using silicon carbide fibers, an Agrobacteria-mediated transformation method, a transformation method using PEG, dextran sulfate, lipofectamine, and drying/inhibition-mediated transformation methods, etc. may be used.

Still another aspect of the present invention provides a method of producing an antibody or a fragment thereof, which includes culturing the transformed cells. Specifically, the production method includes the steps of: i) obtaining a culture by culturing the transformed cells; and ii) recovering the antibody or fragment thereof from the culture.

The method of culturing the transformed cells may be performed using a method well known in the art. Specifically, the culture may be continuously cultured by a batch process, a fed batch process, or a repeated fed batch process.

The step of recovering the antibody or fragment thereof from the culture may be performed by a method known in the art. Specifically, the recovery method includes centrifugation, filtration, extraction, spraying, drying, distillation, precipitation, crystallization, electrophoresis, fractional dissolution (e.g., ammonium sulfate precipitation), chromatography (e.g., ion exchange, affinity, hydrophobicity, and size exclusion), etc. may be used.

Still another aspect of the present invention provides a use of the antibody or fragment thereof for preventing or treating cancer.

Still another aspect of the present invention provides a use of the antibody or fragment thereof for preparing a medicament for preventing or treating cancer.

Still another aspect of the present invention provides a method for preventing or treating cancer which includes administering the antibody or fragment thereof to an individual.

### MODE FOR CARRYING OUT THE INVENTION

Hereinafter, the present disclosure will be described in more detail by the following examples. However, the following examples are for illustrative purposes only, and the scope of the present disclosure is not limited thereto.

### I. Confirmation of Binding Structures of CEACAM1 and Anti-CEACAM1 Antibody

### Example 1. Production of N-domain of CEACAM1

In order to express the amino acid sequence at positions 35 to 141 corresponding to the N-domain of CEACAM1 by binding to a malose-binding protein (MBP) tag to which 10 histidines are linked, the amino acid sequence was cloned into the 10His-MBP-TEV-X-pJA vector derived from the pET28-MBP-TEV vector (Addgene, Plasmid #69929). E. coli strain BL21(DE3)RIPL was transformed using the cloned vector, and the protein was expressed by treating with isopropyl β-D-1-thiogalactopyranoside (IPTG, 200 μM) at 18° C. The E. coli, in which the protein was expressed, was sonicated in a buffer of 100 mM NaCl and 20 mM Tris-HCl (pH 7.5), centrifuged, and only the lysate was separated therefrom. Thereafter, affinity chromatography for the 10His tag was performed using cobalt resin. The eluate obtained therefrom was treated with TEV protease to cleave the 10His-MBP tag. The 10His-MBP tag was separated and removed using a HitrapQ anion exchange column and a Histrap affinity chromatography column, and thereby the N-domain of CEACAM1 with high purity was purified.

### Example 2. Preparation of Fab of Anti-CEACAM1 Antibody

### Example 2.1. Preparation of Anti-CEACAM1 Antibody in which Heavy Chain is Substituted with IgG1 Type

In order to prepare a Fab of an anti-CEACAM1 antibody, the IgG4 heavy chain of the anti-CEACAM1 antibody (SEQ ID NOS: 12 and 13) was converted to human IgG1 (SEQ ID NOS: 14 and 15). For conversion into a human IgG1 type, a gene encoding a CH1-hinge-CH2-CH3 fragment of a human IgG1 type was obtained through PCR. The primers used at this time are shown in Table 2 below.

TABLE 2

| | | SEQ |
|---|---|---|
| Primer | Sequence Information (5′ −> 3′) | ID NO |
| VH-NotI-L_F | GCG GCC GCC ATG TAC TTG GGA CTG | 16 |
| primer | AAC TAT GTA TTC ATA GTT TTT CTC | |
| | TTA AAT GGT GTC CAG AGT | |
| VH-ApaI- | ATG GGC CCT TGG TGG AGG CTG AGG | 17 |
| CH1_R | AGA CGG TGA C | |
| primer | | |
| CH1-Fc_ | GCC TCC ACC AAG GGC CCA | 18 |
| ApalI_F | | |
| primer | | |
| IgG1_Fc_ | NNN NGG ATC CAA GCT TAC TAT TTA | 19 |
| HindIII_R | CCC GGA | |
| primer | | |

A gene encoding the heavy chain of the anti-CEACAM1 antibody-IgG1 type was obtained through overlap PCR of the gene encoding the CH1-hinge-CH2-CH3 fragment of the IgG1 type obtained through the PCR and the gene encoding the heavy chain variable region of the anti-CEACAM1 antibody using primers having restriction sites for NotI (NEB, Cat. No. R0189L) and HindIII (NEB, Cat. No. R0104T). In particular, the primers used are shown in Table 3 below.

TABLE 3

| | | SEQ |
|---|---|---|
| Primer | Sequence Information (5′ −> 3′) | ID NO |
| IgG1_HC_ | GCG GCC GCC ATG TAC TTG G | 20 |
| NotI_F | | |
| primer | | |
| IgG1_HC_ | NNN NGG ATC CAA GCT TAC TAT TTA | 19 |
| HindIII_ | CCC GGA | |
| R primer | | |

The gene (DNA) obtained by the PCR was loaded onto a 1% agarose gel and separated using a DNA Gel extraction kit (QIAGEN, Cat. No. 28706).

Restriction enzymes NotI (NEB, Cat. No. R0189L) and HindIII (NEB, Cat. No. R0104T) were added to the isolated DNA and reacted at 37° C. for 4 hours, and then DNA was obtained using a QIAquick PCR Purification Kit (QIAGEN, Cat. No. 28106).

In addition, the obtained DNA and T4 DNA ligase (NEB, Cat. No. M0203S) were added to a linearized pCIW vector treated with NotI and HindIII restriction enzymes, and reacted at a temperature of 16° C. for 4 hours. After 4 hours, 1 μL of the ligation mixture was taken, added to 100 μL of XL1-Blue electroporation-competent cells, mixed, and transformed using an electroporation system.

In the plate where the transformation occurred, single colonies were inoculated into a SB/car medium and cultured overnight. DNA was obtained from the transformed cells using a QIAprep Spin Miniprep Kit (QIAGEN, Cat. No. 127106), and sequencing was requested to an external company, Cosmogenetech. As a result, a gene encoding an anti-CEACAM1 antibody-IgG1 type heavy chain represented by the nucleotide sequence represented by SEQ ID NO: 33 was confirmed. After culturing the confirmed transformed cells, a large amount of DNA was obtained using the QIAGEN Plasmid Plus Midi Kit (QIAGEN, Cat. No. 12945).

### Example 2.2. Production of Anti-CEACAM1 antibody in which heavy chain is substituted with IgG1 type

Expi293F™ Cells (Gibco, Cat. No. A14527) were seeded one day before transfection at a concentration of 2.0×10⁶ cells/mL, in Expi293™ Expression Medium (Gibco, Cat. No. A1435101). After incubation under the conditions of 37° C., 8% CO₂, and 125 rpm for 24 hours, 25.5 mL was prepared at a concentration of 2.5×10⁶ cells/mL (viability=95%).

30 μg of the heavy chain DNA (15 μg of pC1W_MG1124HC_IgG1 and 15 μg of pCIW_MG1124LC) of the obtained CEACAM1 antibody-IgG1 type was diluted in 1.5 mL of the OptiPro™ SEM medium (Gibco, Cat. No. 12309019) and reacted at room temperature for 5 minutes. Then, 80 μL of ExpiFectamine™ 293 reagent (Gibco, Cat. No. A14524) was also added into 1.5 mL of the OptiPro™ SEM medium (Gibco, Cat. No. 12309019) and reacted at room temperature for 5 minutes. Thereafter, the respective diluted DNA and ExpiFectamine™ 293 reagent were well mixed, and 3 mL of the mixture was reacted at room temperature for 30 minutes.

3 mL of the mixture was added to 30 mL of Expi293F™ cells at a concentration of 2.5×10⁶ cells/mL (viability=95%). After incubating the mixture for 16 to 18 hours, 150 μL of ExpiFectamine™ 293 enhancer 1 (Gibco, Cat. No. A14524) and 1.5 μL of ExpiFectamine™ 293 enhancer 2 (Gibco, Cat. No. A14524) were added thereto, and cultured in a CO₂ shaking incubator (MB-206CXXL) for 6 days.

After the culture was completed, the cells were centrifuged at 4,000 rpm for 20 minutes and the cell pellet was removed and the supernatant was filtered through a 0.45 μm filter. 100 μL of CaptivA Protein A resin (REPLIGEN, CA-PRI-0100), which is a Protein A resin, was prepared per 30 mL of each culture, centrifuged at 1,000 rpm for 2 minutes to remove the storage buffer, and washed 3 times with 1 mL of Protein A binding buffer (Pierce, Cat. No. 21007) for each wash.

Protein A resin was added into the prepared culture solution. After performing rotating incubation at room temperature for 2 hours, the mixture was added to the Pierce Spin column snap-cap (Thermo, Cat. No. 69725) and QIAvac 24 plus (QIAGEN, Cat. No. 19413), and the column was filled with resin using a vacuum manifold. The resin was washed by adding 5 mL of Protein A binding buffer (Pierce, Cat. No. 21007) thereto, and 200 μL of Protein A elution buffer (Pierce, Cat. No. 21009) was added thereto. The mixture was incubated for 2 minutes at room temperature, and then eluted by centrifugation under a 1,000 rpm condition for 1 minute.

### Example 2.3. Isolation and Purification of Fab of Anti-CEACAM1 antibody

In order to purify only the Fab from the anti-CEACAM1 antibody-IgG1 type prepared in Example 2.2, papain protease was treated thereon at a ratio of 1:100. Papain protease was used to separate Fab and Fc by cleaving the sequence between the Fab and Fc of the IgG1 heavy chain. The papain protease treatment was reacted in PBS buffer, and then the buffer conditions were converted by dialysis with 0 mM NaCl and 20 mM Tris-HCl (pH 7.5). Since the Fab portion of the anti-CEACAM1 antibody had a theoretical pI value of 8.8, it was separated and purified using a HitrapSP cation exchange column. The Fc of anti-CEACAM1 was released without being attached to the column, and the Fab of the anti-CEACAM1 was separated and purified with high purity under the condition of about 50 mM to about 70 mM NaCl. The purified anti- CEACAM1 Fab and the CEACAM1 N-domain separated in Example 1 were mixed at a 1:2 ratio and finally purified through a HiLoad 26/60 Superdex 75 gel-filtration column.

### Example 3. Crystallization and Structural Analysis of Fab Complex of CEACAM1 N-domain and Anti-CEACAM1 antibody

The crystallization conditions were optimized by screening under about 800 conditions.

Finally, the complex of a Fab of the anti-CEACAM1 antibody and the N-domain of the CEACAM1 (36.6 mg/mL) was crystallized under the condition where 0.1 M lithium sulfate monohydrate, 0.1 M N-(2-acetamido)iminodiacetic acid (ADA) (pH 6.5), 14% (w/v) polyethylene glycol 4000, and 2% (v/v) isopropanol. The resulting crystals were treated under conditions, where 17.5% ethylene glycol was additionally, for prevention of being frozen.

X-ray diffraction (XRD) data were collected in the 5C beamline at Pohang Accelerator Laboratory and processed with HKL2000 suit (1). The structure of the complex was determined using the Molecular replacement (2) method of the Phenix program, and the model used for the same was a structure having high sequence homology with an anti-CEACAM antibody (PDB entry: 4EVN) and a structure corresponding to the CEACAM1 N-domain. (PDB entry: 4WHD). For the refinement of the structure, Refinement function of Phenix (3), CNS program (4), and COOT program (5) were used. Crystallographic data statistics are shown in Table 4 below.

TABLE 4
Data Collection

| | |
|---|---|
| Space Group | P212121 |

Unit Cell Dimensions

| | |
|---|---|
| a, b, c (Å) | 70.985, 140.722, 206.120 |
| α, β, γ (°) | 90, 90, 90 |
| Wavelength (Å) | 1.0000 |
| Resolution (Å) | 50.0-1.79 |
| R_{sym} | 9.9 (27.0) |
| I/(I) | 14.6 (1.9) |
| Completeness (%) | 73.8 (23.8) |
| Redundancy | 5.6 (1.3) |

Refinement

| | |
|---|---|
| Resolution (Å) | 50.0-1.8 |
| No. of Reflections | 142860 |
| R_{work}/R_{free} (%) | 22.83/25.76 |

R.m.s deviations

| | |
|---|---|
| bond lengths (Å)/angles (°) | 0.008/0.979 |
| Average B-Values (Å²) | 29.6 |
| Ramachandran Plot (%) | |
| Favored/Additional Allowed | 89.8/10.0 |
| Generously Allowed | 0.0 |

^{a}The numbers in parentheses are the statistics from the highest resolution shell.

After undergoing the crystallization process, XRD data were obtained, and the structure of the complex of the Fab of the anti-CEACAM1 antibody and the CEACAM1 N-domain with a resolution of 1.8 Å was finally identified. Based on the crystal structure, the paratope of the Fab of the anti-CEACAM1 antibody that binds within an intermolecular distance of 4.5 Å and the epitope of the CEACAM1 N-domain were identified.

As a result, the heavy chain variable region of the anti-CEACAM1 antibody showed a higher number of bindings to CEACAM1 than the light chain variable region. In the case of the heavy chain variable region, the amino acids Asn31, Tyr32, Gly54, Gly56, Ser57, Asn74, Pro100, Thr101, Lys102, Tyr104, and Ala 105 were hydrophobically or electrostatically bound to the CEACAM1 N-domain. In the case of the light chain variable region, the amino acids Tyr33, Tyr50, Ala51, and Leu94 were hydrophobically or electrostatically bound to the CEACAM1 N-domain.

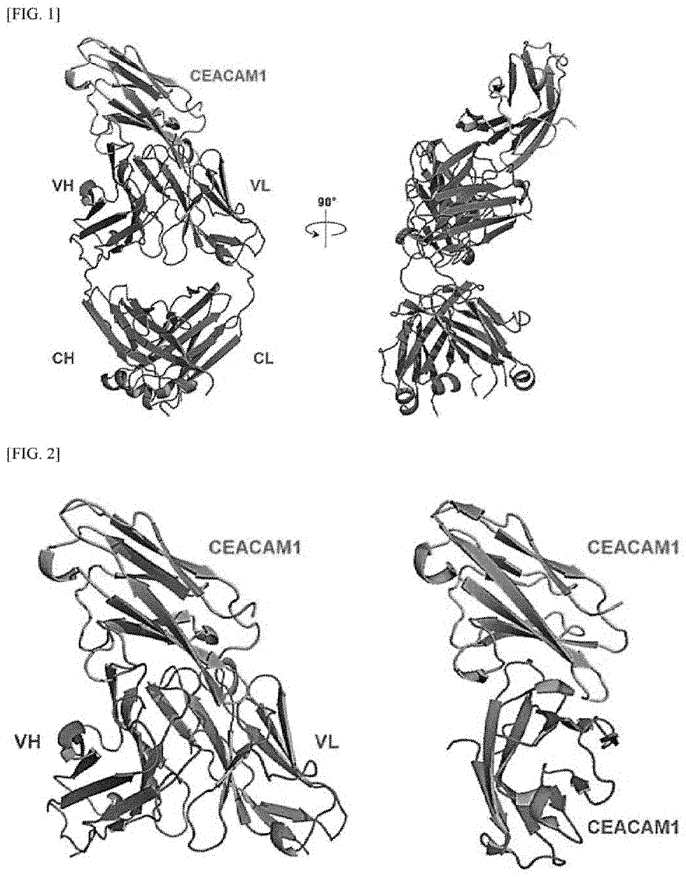

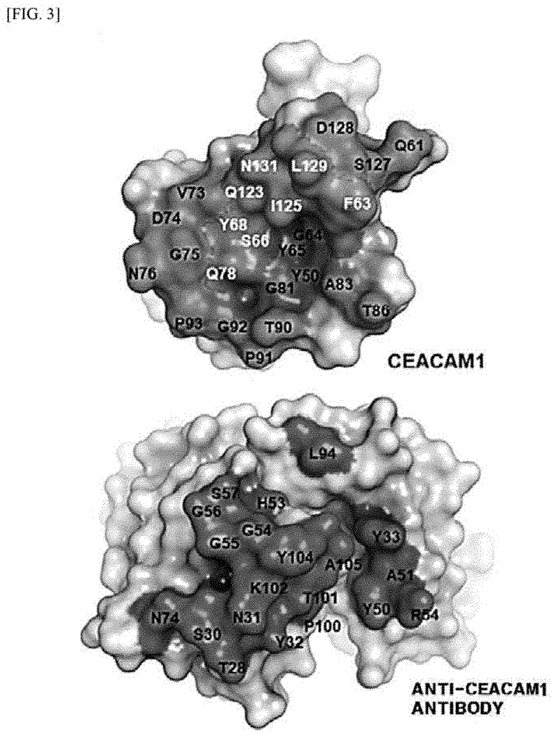

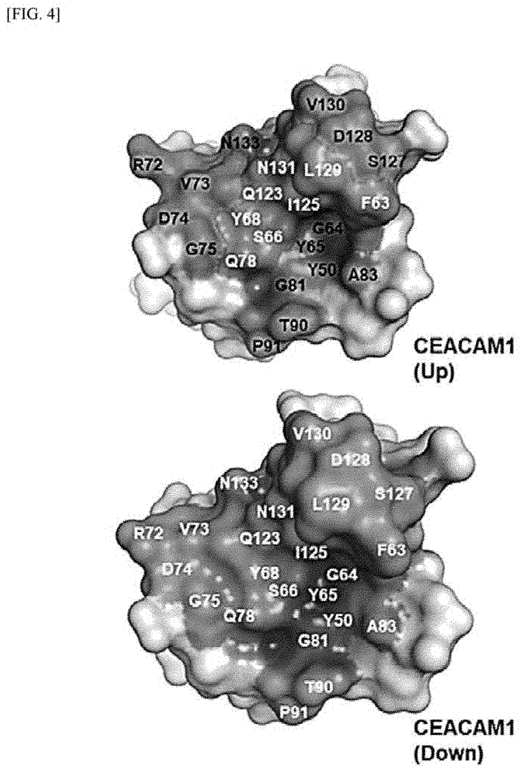

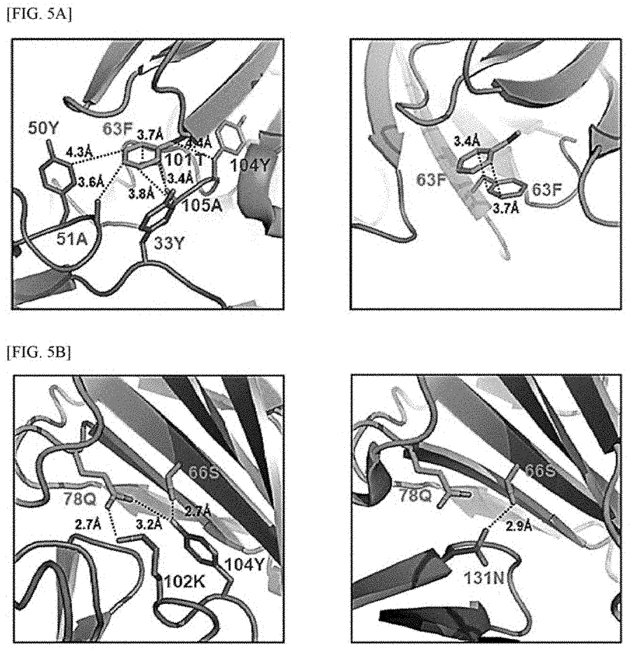

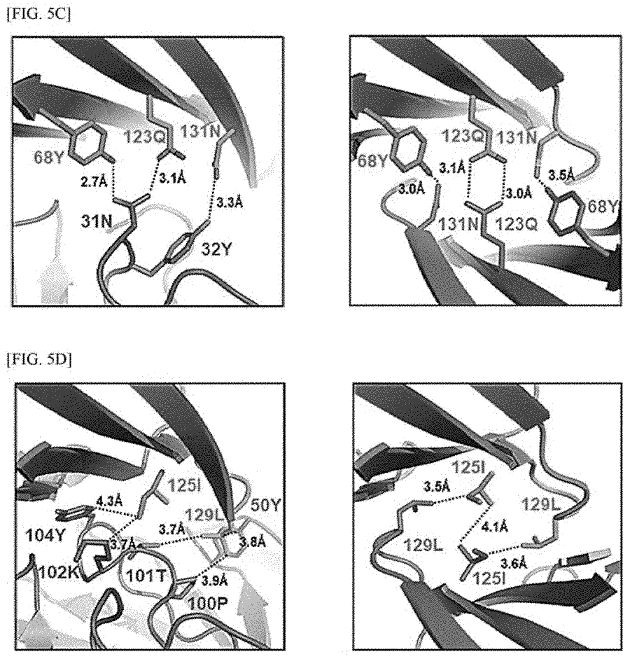

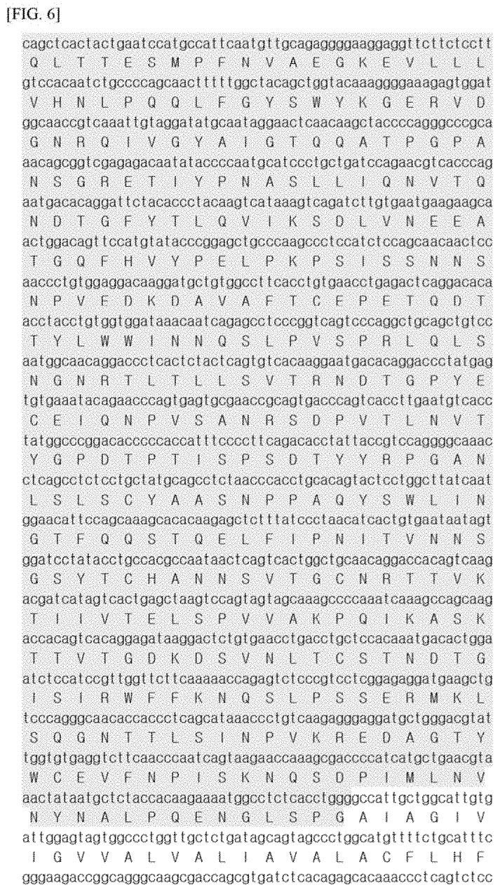

Based on the CEACAM1N-domain, the amino acids Phe63, Gly64, Ser66, Tyr68, Gly75, Asn76, Gln78, Ala83, Thr86, Thr90, Gln123, Ile125 Leu129, and Asn131 were bound to the anti-CEACAM 1 antibody. The total surface area of antigen-antibody binding was 791.98 Å² (FIGS. 1 and 2). The amino acids that play an important role in forming a dimer of the previously known CEACAM1 N-domain are Phe63, Ser66, Tyr68, Gln78, Gln123, Ile125, Leu129, and Asn131 and are shown in FIGS. 3 and 4. This confirms that the CEACAM1 N-domain can also be bound by the Fab of the anti-CEACAM1 antibody (FIGS. 5A to 5D). From these results, it was confirmed that the epitope of the CEACAM1 N-domain, to which the anti-CEACAM1 antibody binds, overlaps with the dimer interface of CEACAM1, and thus anti-CEACAM1 CEACAM1 can effectively inhibit the activity of CEACAM1.

### Experimental Example 4. Measurement of Binding Strength between Anti-CEACAM1 antibody and CEACAM1

Quantitative binding strength of the anti-CEACAM1 antibody to CEACAM1 was measured using the Octet QKe (Pall ForteBio). The anti-CEACAM1 antibody was subjected to a ½ concentration dilution 6 times at a concentration of 400 nM, and the antibodies at concentrations of 400 nM, 200 nM, 100 nM, 50 nM, 25 nM, 12.5 nM, and 6.25 nM were added into a Greiner 96-well-plate (Greiner, Cat. No. 655209) in an amount of 200 μL each in a row, and the last well was added at a concentration of 0 nM. In another row, hCEACAM1 (R&D Systems, Cat. No. 2244-CM) was diluted to a concentration of 6.25 μg/μL, and was added in an amount of 200 μL each in a row.

Washing buffer, neutralization buffer, and baseline buffer were diluted 10 times with Reagent/Kinetics buffer (10×) (Fortebio, Cat. No. 18-1092) and was added in an amount of 200 μL in each row, and regeneration buffer was added in an amount of 200 μL in a row. A separate Greiner 96-well-plate was prepared, and Reagent/Kinetics buffer (IX) was added in an amount of 200 μL to as many wells as the number of biosensors to be used, and the Biosensors/Anti-His(His1K) (Fortebio, Cat. No. 18)-5120) Cassette was mounted.

The time for association and dissociation was each set to 300 seconds and 600 seconds and their KD values were measured.

As a result, as shown in Table 5 below, it was confirmed that the anti-CEACAM1 antibody had an affinity (KD) value of 3.90 nM for CEACAM1.

TABLE 5

| K_{D} (M) | Kₒₙ (1/Ms) | K_{off} (1/s) |
|---|---|---|
| 3.39 × 10⁻⁹ | 1.63 × 10⁵ | 5.67 × 10⁻⁴ |

### II. Confirmation of Activity of Anti-CEACAM1 Antibody

### Example 5.1. Preparation of hCEACAM1-Fc

The extracellular domain (Gln35-Gly428) of CEACAM1 from human CEACAM1 cDNA (R&D systems, RDC0951) was subjected to PCR using a NotI-SS-CEACAM1 forward primer and a CEACAM1-Hinge reverse primer. In particular, the primers used are shown in Table 6 below.

TABLE 6

| | | SEQ |
|---|---|---|
| Primer | Sequence Information (5′ −> 3′) | ID NO |
| NotI-SS- | GCG GCC GCC ATG TAC TTG GGA CTG | 21 |
| CEACAM1 | AAC TAT GTA TTC ATA GTT TTT CTC | |
| F primer | TTA AAT GGT GTC CAG AGT CAG CTC | |
| | ACT ACT GAA TCC ATG | |
| CEACAM1- | GCA AGG AGG GCC GTA CTT AGA CTC | 22 |
| Hinge R | CCC AGG TGA GAG GCC ATT TTC | |
| primer | | |

The DNA obtained through the PCR was loaded on a 1% agarose gel, and DNA encoding the hCEACAM1 extracellular domain (FIG. 6 and SEQ ID NO: 23) was obtained using a DNA Gel extraction kit.

In addition, to obtain the gene of the human IgG4 Fc region, PCR was performed using the heavy chain DNA of the anti-CEACAM1 antibody of SEQ ID NO: 12 as a template and using a hIgG4-Hinge forward primer and a hlgG4-CH3-stop-HindIII reverse primer. In particular, the primers used are shown in Table 7 below.

TABLE 7

| | | SEQ |
|---|---|---|
| Primer | Sequence Information (5′ −> 3′) | ID NO |
| hIgG4- | GAG TCT AAG TAC GGC CCT CC T TGC | 24 |
| Hinge | | |
| F primer | | |
| hIgG4-CH3- | GGA TCC AAG CTT ACT ACT TTC CCA | 25 |
| stop | GTG ACA GTG A | |
| HindIII R | | |
| primer | | |

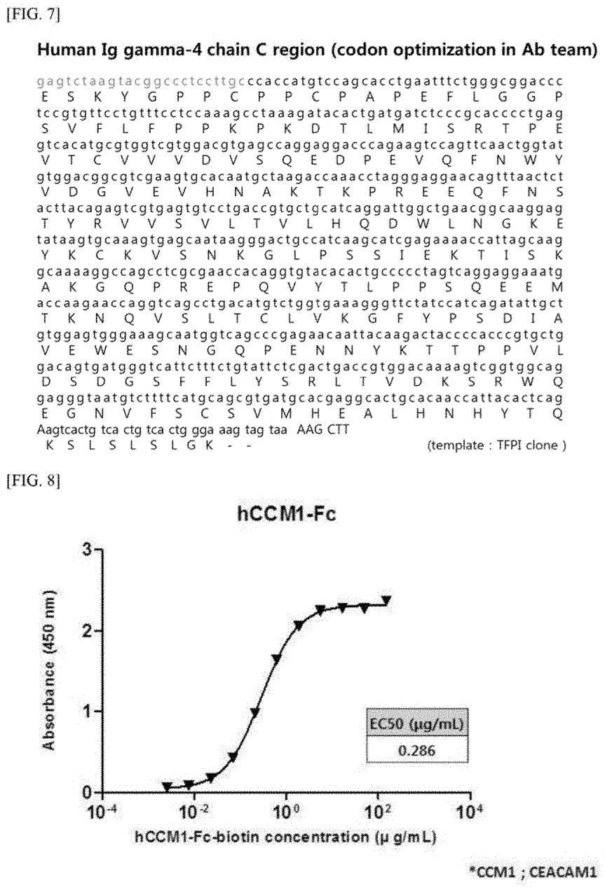

DNA obtained through PCR was loaded on a 1% agarose gel, and DNA encoding the hlgG4 Fc region (FIG. 7 and SEQ ID NO: 26) was obtained using a DNA Gel extraction kit. The PCR was performed using the DNA encoding the hCEACAM1 extracellular domain and the DNA encoding the hlgG4 Fc region using a NotI-SS-CEACAM1 forward primer and a hIgG4-CH3-stop HindIII reverse primer with restriction enzyme cleavage sites.

DNA obtained through PCR was loaded on a 1% agarose gel, and hCEACAM1-Fc DNA was obtained using a DNA Gel extraction kit. The obtained hCEACAM1-Fc DNA was treated with Notl and HindIII restriction enzymes and reacted at 37° C. for 4 hours, and then hCEACAM 1-Fc insert DNA fragments were isolated using a QIAquick PCR Purification Kit. Thereafter, in order to clone the prepared hCEACAM1-Fc insert DNA fragments into a pcIW vector, the pcIW vector was also separated into a linear pCIW vector by treatment with NotI and HindIII restriction enzymes. T4 DNA ligase were added to the HCEACAM1-Fc insert DNA fragments digested with NotI and HindIII restriction enzymes and the linear pcIW vector and reacted at 16° C. for 4 hours. After 4 hours, 1 μL of the ligation mixture was collected, added into 100 μL of XL1-Blue electroporation-competent cells, mixed, and transformed using an electroporation method.

In the transformed plate, single colonies were inoculated into the SB/car medium and cultured overnight. DNA was obtained from the transformed cells using the QIAprep Spin Miniprep Kit (QIAGEN, Cat. No. 127106), and sequencing was requested to an external company, Cosmogenetech. As a result, a gene encoding hCEACAM1-Fc represented by a nucleotide sequence of SEQ ID NO: 34 was identified. After culturing the identified transformed cells, a large amount of DNA was obtained using the QIAGEN Plasmid Plus Midi Kit (QIAGEN, Cat. No. 12945).

Thereafter, the hCEACAM1-Fc protein was produced and isolated by a transient overexpression system using Expi293F animal cells in the same manner as in Example 2.2.

### Example 5.2. Confirmation of Interaction between hCEACAM1-Fc and hCEACAM1-Fc

The hCEACAM1-Fc prepared in Example 5.1 was diluted with PBS to a concentration of 2.5 μg/mL, and then 100 μL each of the resultant was added into a NUNC immuno module (NUNC, 468667) plate and coated at 4° C. overnight. On the next day, after washing with PBS (to which 0.05% Tween 20 was added), 300 μL of PBS (to which 1% BSA was added) was added to each well of the coated plate, and blocking was performed by reacting at room temperature for 2 hours. In addition, after washing 3 times with PBS (to which 0.05% Tween 20 was added), biotin-labeled hCEACAM1-Fc was serially diluted 3-fold starting from a concentration of 150 μg/mL, and added into 11 wells in the plate coated with hCEACAM1-Fc. The last well was added only with PBS to which 1% BSA was added. The wells were reacted at 37° C. for 1 hour. After 1 hour, each well was washed 3 times by adding 300 μL of PBS (to which 0.05% Tween 20 was added), and then streptavidin-peroxidase polymer (Sigma, S2438-250UG) was diluted at a 1:5,000 ratio in PBS (to which 1% BSA was added), 100 μL each of the resultant was added into each well, and reacted at 37° C. for 40 minutes. Each well where the reaction was completed was washed 3 times by adding 300 μL of PBS (to which 0.05% Tween 20 was added) thereto, and then 100 μL of TMB microwell peroxidase substrate (KPL, 50-76-03) was added to each well, and reacted for 5 minutes. To terminate the reaction, an equal amount of sulfuric acid (Sigma-Aldrich, 339741) was added and the OD value was measured at 450 nm wavelength using an ELISA reader.

As a result, it was confirmed that the binding depends on the concentration of the hCEACAM 1-Fc protein. In particular, the EC50 value was 0.286 μg/mL (FIG. 8).

### Example 5.3. Confirmation of Effect of Anti-CEACAM1 antibody on Inhibition of hCEACAM1-hCEACAM1 Interaction

The hCEACAM1-Fc prepared in Example 5.1 was diluted with PBS to a concentration of 2.5 μg/mL, and then 100 μL each of the resultant was added into a NUNC immuno module (NUNC, 468667) plate and coated at 4° C. overnight. On the next day, 300 μL of PBS (to which 1% BSA was added) was added to each well in the coated plate and blocking was performed by reacting at room temperature for 2 hours.

In another 96-well-plate, anti-CEACAM1 antibody was serially diluted 2-fold starting from a concentration of 60 μg/ml, and added into 11 wells, and the last well was added only with PBS to which 1% BSA was added, and then biotin-labeled hCEACAM1-Fc was added in the same amount as the anti-CEACAM1 antibody (which was diluted to 0.5 μg/mL), and reacted at room temperature for 1 hour. After 1 hour, 100 μL each of a mixture of the anti-CEACAM1 antibody and the biotin-labeled hCEACAM1-Fc were added to the blocked plate and reacted again at room temperature for 1 hour.

After 1 hour, each well was washed 3 times by adding 300 μL of PBS (to which 0.05% Tween 20 was added) thereto, and then streptavidin-peroxidase polymer (Sigma, S2438-250UG) was diluted at a 1:5,000 ratio in PBS (to which 1% BSA was added), 100 μL each of the resultant was added to each well, and reacted at 37° C. for 40 minutes. Each well where the reaction was completed was washed 3 times by adding 300 μL of PBS (to which 0.05% Tween 20 was added), and then 100 μL of TMB microwell peroxidase substrate (KPL, 50-76-03) was added thereto and reacted for 5 minutes. To terminate the reaction, an equal amount of sulfuric acid (Sigma-Aldrich, 339741) was added and the OD value was measured at 450 nm wavelength using an ELISA reader.

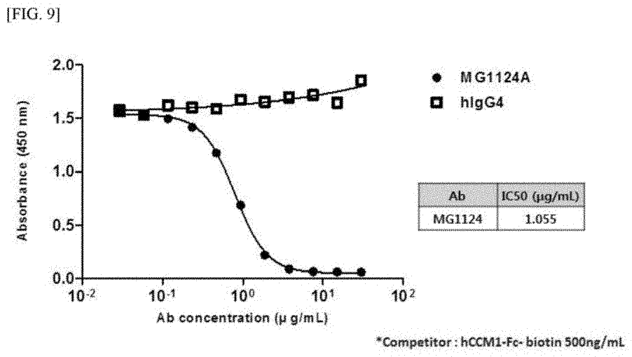

As a result, it was found that the homophilic interaction of hCEACAM1-Fc was inhibited as the concentration of the anti-CEACAM1 antibody increased. In particular, the IC50 value was 1.055 μg/mL (FIG. 9).

### Example 6.1. Preparation of hCEACAM6-Fc

The extracellular domain (Lys35-Gly320) of CEACAM6 from human CEACAM6 cDNA (R&D Systems, RDC0955) was subjected to PCR using a SS-hCEACAM6 forward primer and a CEACAM6-Hinge reverse primer. In particular, the primers used are shown in Table 8 below.

TABLE 8

| | | SEQ |
|---|---|---|
| Primer | Sequence Information (5′ −> 3′) | ID NO |
| SS hCEACAM6 | AAT GGT GTC CAG AGT AAG CTC ACT | 27 |
| F | ATT GAA TCC | |
| hCEACAM6_R_ | GTC ACA AGA TTT GGG CTC TCC AGA | 28 |
| hinge(HIgG1) | GAC TGT GAT CAT | |

The DNA obtained through the PCR was loaded on a 1% agarose gel, and the DNA encoding the hCEACAM6 extracellular domain was obtained using a DNA Gel extraction kit.

In addition, in order to obtain the gene of the human IgG1 Fc region, PCR was performed using a heavy chain vector of an anti-CEACAM1 antibody-IgG1 type as a template using a hinge (HIgG1) forward primer and a CH3 (HIgG1)_HindIII reverse primer. In particular, the primers used are shown in Table 9 below.

TABLE 9

| | | SEQ |
|---|---|---|
| Primer | Sequence Information (5′ −> 3′) | ID NO |
| hinge(HIgG1)_ | GAG CCC AAA TCT TGT GAC | 29 |
| F primer | TTG GAT CCA AGC TTA CTA TTT ACC | |
| CH3(HIgG1)_ | CGG AGA CAG GGA | 30 |
| HindIII_R | | |
| primer | | |

The DNA obtained through the PCR was loaded on a 1% agarose gel, and the DNA encoding the hIgG1 Fc region was obtained using a DNA Gel extraction kit. The DNA encoding the hCEACAM6 extracellular domain and the DNA encoding the hIgG1 Fc region were subjected to PCR using a NotI-SS-forward primer with a restriction enzyme cleavage site and a CH3(HIgG1)_HindIII reverse primer. In particular, the primers used are shown in Table 10 below.

TABLE 10

| | | SEQ |
|---|---|---|
| Primer | Sequence Information (5′ −> 3′) | ID NO |
| NotI-SS_ | NNN NNN GCG GCC GCC ATG TAC TTG | 31 |
| F primer | GGA CTG AAC TAT GTA TTC ATA GTT | |
| | TTT CTC TTA AAT GGT GTC CAG AGT | |
| CH3(HIgG1)_ | TTG GAT CCA AGC TTA CTA TTT ACC | 30 |
| HindIII_R | CGG AGA CAG GGA | |
| primer | | |

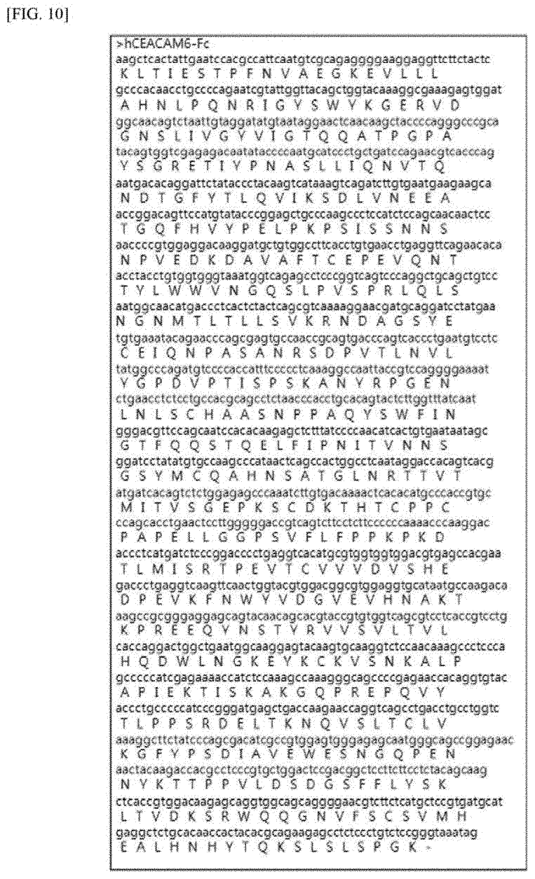

The DNA obtained through the PCR was loaded on a 1% agarose gel, and a DNA encoding hCEACAM6 (ECD)-Fc was obtained using a DNA Gel extraction kit (FIG. 10 and SEQ ID NO: 32). The obtained hCEACAM6-Fc DNA was treated with NotI and HindIII restriction enzymes and reacted at 37° C. for 4 hours, and then hCEACAM6-Fc insert DNA fragment was isolated using a QIAquick PCR Purification Kit. Thereafter, in order to clone the prepared hCEACAM6-Fc insert DNA fragment into a pcIW vector, the pcIW vector was also isolated into a linear pCIW vector by treating with NotI and HindIII restriction enzymes. T4 DNA ligase was added to the hCEACAM6-Fc insert DNA fragment and the linear pcIW vector digested with NotI and HindIII restriction enzymes, and reacted at 16° C. for 4 hours. After 4 hours, 1 μl, of the ligation mixture was collected, added into 100 μL of XL1-Blue electroporation-competent cells, mixed, and transformed using an electroporation method.

In the transformed plate, single colonies were inoculated into a SB/car medium and cultured overnight. DNA was obtained from the transformed cells using the QIAprep Spin Miniprep Kit (QIAGEN, Cat. No. 127106), and sequencing was requested to an external company, Cosmogenetech. As a result, a gene encoding hCEACAM6-Fc was identified. After culturing the identified transformed cells, a large amount of DNA was obtained using the QIAGEN Plasmid Plus Midi Kit (QIAGEN, Cat. No. 12945).

Thereafter, the hCEACAM6-Fc protein was produced and isolated by a transient overexpression system using Expi293F animal cells in the same manner as in Example 2.2.

### Example 6.2. Confirmation of Interaction between hCEACAM1-Fc and hCEACAM6-Fc

The hCEACAM6-Fc prepared in Example 6.1 was diluted with PBS to a concentration of 2.5 μg/mL, and then 100 μL each of the resultant was added into a NUNC immuno module (NUNC, 468667) plate and coated at 4° C. overnight. On the next day, 300 μL of PBS (to which 1% BSA was added) was added each well in the coated plate and blocking was performed by reacting at room temperature for 2 hours.

In addition, biotin-labeled hCEACAM1-Fc was serially diluted 3-fold starting from a concentration of 150 μg/mL and added into 11 wells in the plate coated with hCEACAM6-Fc, and the last well was added with only PBS (to which 1% BSA was added) and reacted at a temperature of 37° C. for 1 hour. After 1 hour, each well was washed 3 times by adding 300 μL of PBS (to which 0.05% Tween 20 was added) thereto, and then streptavidin-peroxidase polymer (Sigma, S2438-250UG) was diluted at a 1:5,000 ratio in PBS (to which 1% BSA was added), and 100 μL each of the resultant was added to each well, and reacted at 37° C. for 40 minutes. Each well where the reaction was completed was washed 3 times by adding 300 μL of PBS (to which 0.05% Tween 20 was added) thereto, and then 100 μL of TMB microwell peroxidase substrate (KPI., 50-76-03) was added thereto, and reacted for 5 minutes. To terminate the reaction, an equal amount of sulfuric acid (Sigma-Aldrich, 339741) was added and the OD value was measured at 450 nm wavelength using an ELISA reader.

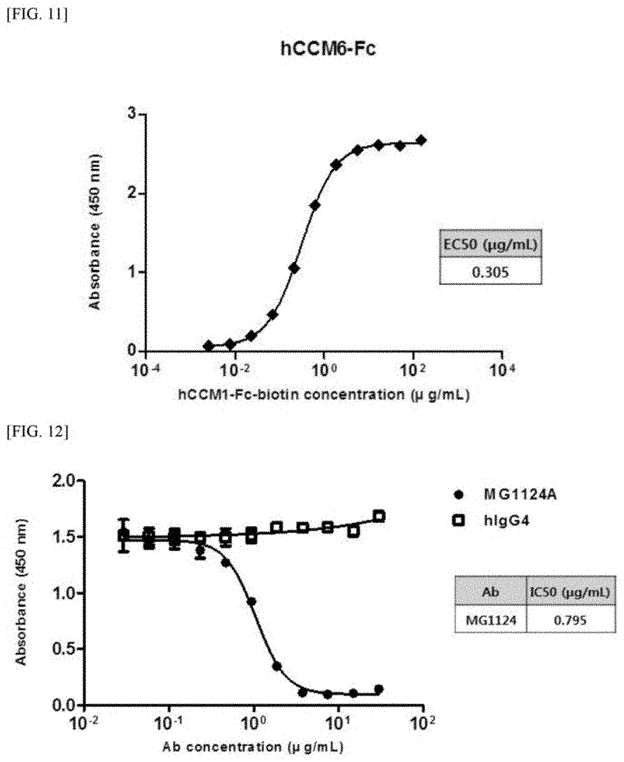

As a result, it was confirmed that it bound to hCEACAM6-Fc as the concentration of the hCEACAM1-Fc protein increased. In particular, the EC50 value was 0.305 μg/mL (FIG. 11).

### Example 6.3. Confirmation of Effect of Anti-CEACAM1 Antibody on Inhibition of hCEACAM1-hCEACAM1 Interaction

The hCEACAM6-Fc prepared in Example 6.1 was diluted with PBS to a concentration of 2.5 μg/mL, and then 100 μL each of the resultant was added into a NUNC immuno module (NUNC, 468667) plate and coated at 4° C. overnight. On the next day, 300 μl of PBS (to which 1% BSA was added) was added to each well in the coated plate and blocking was performed by reacting at room temperature for 2 hours.

In another 96-well-plate, the anti-CEACAM1 antibody was serially diluted 2-fold starting from a concentration of 60 μg/mL and added into 11 wells, and the last well was added with only PBS (to which 1% BSA was added), and then biotin-labeled hCEACAM 1-Fc was added in the same amount as the diluted anti-CEACAM1 antibody to a concentration of 0.5 μg/mL, and reacted at room temperature for 1 hour. After 1 hour, 100 μL each of a mixture of the anti-CEACAM1 antibody and the biotin-labeled hCEACAM1-Fc were added to the blocked plate and reacted again at room temperature for 1 hour.

After 1 hour, each well was washed 3 times by adding 300 μL of PBS (to which 0.05% Tween 20 was added) thereto, and then streptavidin-peroxidase polymer (Sigma, S2438-250UG) was diluted at a 1:5,000 ratio in PBS (to which 1% BSA was added), and 100 μL each of the resultant was added to each well, and reacted at 37° C. for 40 minutes. Each well where the reaction was completed was washed 3 by adding 300 μL of PBS (to which 0.05% Tween 20 was added) thereto, and then 100 μL each of TMB microwell peroxidase substrate (KPL, 50-76-03) was added thereto, and reacted for 5 minutes. To terminate the reaction, an equal amount of sulfuric acid (Sigma-Aldrich, 339741) was added and the OD value was measured at 450 nm wavelength using an ELISA reader. As a result, it was found that the binding between hCEACAM1-Fc and hCEACAM6-Fc was inhibited as the concentration of the anti-CEACAM1 antibody increased. In particular, the IC50 value was 0.795 μg/mL (FIG. 12).

### III. Confirmation of Effect of Anti-CEACAM1 Antibody on Activation of TCR Signaling

In order to confirm whether the anti-CEACAM1 antibody blocks the inhibition of T cell activation due to the CEACAM1-CAECAM1 interaction and thereby activates TCR signaling, the presence of phosphorylation of ZAP70, which is one of the TCR signaling pathways, and the increase of nuclear factor of activated T-cells (NFAT) transcription factor and 1L-2 expression levels were examined.

### Example 7.1. Confirmation of Effect of ZAP70 Phosphorylation

First, in order to prepare Jurkat cells overexpressing CEACAM1, a nucleotide sequence encoding CEACAM 1 was inserted into the pEF1α-AcGFP-N1 plasmid (Clontech, Cat. No. 631973) using restriction enzymes and thereby pEF1α-AcGFP-N1-CCM1 plasmid was prepared.

Specifically, the pEF1α-AcGFP-N1-CCM1 plasmid was subjected to PCR from human CEACAM1 cDNA (R&D, Cat. No. RDC0951) using a CEACAM1 forward primer with a HindIII restriction enzyme cleavage site and a reverse primer with a SalI restriction enzyme cleavage site. In particular, the primers used are shown in Table 11 below.

TABLE 11

| | | SEQ |
|---|---|---|
| Primer | Sequence Information (5′ −> 3′) | ID NO |
| CEACAM1_ | GAC AAG CTT ATG GGG CAC CTC TCA | 35 |
| HindIII_ | GCC C | |
| F primer | | |
| CEACAM1_ | GAC GTC GAC GTC TGC TTT TTT ACT | 36 |
| SalI_R | TCT GAA TAA | |
| primer | | |

The DNA obtained through the PCR was loaded on a 0.8% agarose gel, and HindIII/SalI hCEACAM1 DNA was obtained using a DNA gel extraction kit (Promega, Cat. No. A9282). The obtained hCEACAM1 DNA was treated with HindIII and SalI restriction enzymes and reacted at 37° C. for 2 hours, and then the hCEACAM1 insert DNA fragment was isolated using a Gel and PCR clean up system (Promega, Cat. No. A9282). Thereafter, in order to clone the prepared hCEACAM1 insert DNA fragment into the pEF1α-AcGFP-N1 vector (Clontech, Cat No. 631973), the pEF1α-AcGFP-N1 vector was also treated with HindIII and SalI restriction enzymes to isolate a linear pEF1α-AcGFP-N1 vector. T4 DNA ligase was added to the linear pEF1α-AcGFP-N1 vector and the HCEACAM1 insert DNA fragments digested with HindllI and SalI restriction enzymes, and reacted at 16° C. for 4 hours. After 4 hours, 1 μL of the ligation mixture was collected and transformed into 100 μL of competent cells of the DH-5αE. coli strain. In the transformed plate, single colonies were inoculated into the medium and cultured overnight. The transformed cells were obtained using DNA plasmid SV (Geneall, Cat. No. 101-102), and sequencing was requested to an external company, Cosmogenetech.

As a result, a gene encoding hCEACAM1 represented by the nucleotide sequence of SEQ ID NO: 37 was identified. After culturing the identified transformed cells, a large amount of DNA was obtained using the QIAGEN Plasmid Plus Midi Kit (QIAGEN, Cat. No. 12945).

Thereafter, 10 μg of the pEF1α-AcGFP-N1-CCM1 plasmid was transfected into 3×10⁶ Jurkat E6.1 cells (ATCC) using a Neon transfection system (1,400 voltage/20 ms/2 pulse). After 72 hours, the transfected Jurkat cells were harvested, and Jurkat cells that express GFP were sorted using a flow cytometer (FACSAria). The sorted Jurkat cells were cultured in a culture medium containing 1 mg/mL of G418 (Sigma, Cat. No. G8168). In particular, the culture medium used was a complete IMDM (cIMDM) medium containing Pen/Strep (1×), NEAA (1×), sodium pyruvate (1×), and 10% FBS.

Jurkat cells and Jurkat cells overexpressing CEACAM1 were dispensed at a density of 3×10⁶ cells per well, treated with 10 μg/mL of hIgG4 (Sigma, Cat. No. 14639) or anti-CEACAM 1 antibody for 5 hours, and cultured under the conditions of 5% CO₂ at a temperature of 37° C. Thereafter, some cultured Jurkat cells and Jurkat cells overexpressing CEACAM1 were stimulated for 1 minute by treating with 1 μg/mL of anti-CD3 antibody (eBioscience, Cat. No. 16-0037-85) and anti-CD28 antibody (eBioscience, Cat. No. 16-0289-85).

The Jurkat cells and the Jurkat cells overexpressing CEACAM 1 were each lysed using a RIPA buffer (ice-cold lysis buffer). Thereafter, the cell lysates were each centrifuged, mixed with 6× Laemmli buffer, and the mixture was loaded onto a Novex 4-12% Bis-Tris gradient gel using an MES running buffer to transfer the proteins in the mixture onto a nitrocellulose membrane. Thereafter, non-specific reactions were blocked by treating the resultant with 5% bovine serum albumin (BSA), and Western blot was performed using a primary antibody and a secondary antibody conjugated with HRP. In particular, the primary antibodies used were an anti-phosphorylation-ZAP70 (Y319, Cell signaling, Cat. No. 2717S), an anti-ZAP70 antibody (Cell signaling, Cat. No. 2705S), an anti-CEACAM1 antibody (ORIGENE, Cat. No. No. 2717S). TA350817), and an anti-actin antibody (Cell signaling, Cat. No. 49671).

In particular, the Jurkat cells which did not express CEACAM1 and were not stimulated with the anti-CD3 antibody and the anti-CD28 antibody were set as a negative control, and the Jurkat cells which were stimulated with the anti-CD3 antibody and the anti-CD28 antibodt, did not express CEACAM1 and were treated with hIgG4 were set as positive controls. In addition, the Jurkat cells overexpressing CEACAM1, which were stimulated with the anti-CD3 antibody and the anti-CD28 antibody and were treated with hIgG4 or the anti-CEACAM1 antibody were set as the experimental group.

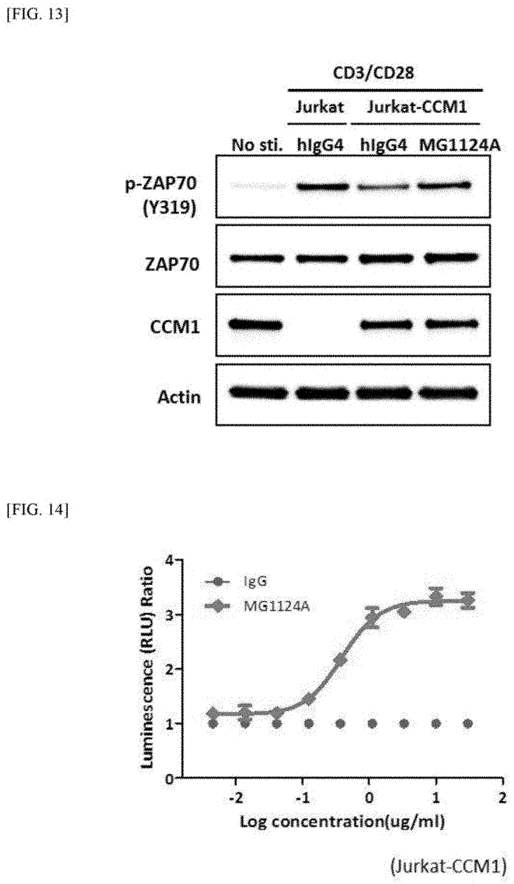

As a result, as shown in FIG. 13, it was confirmed that in the case of the negative control group, ZAP70 was not phosphorylated, whereas in the case of the positive control group, ZAP70 was phosphorylated (p-ZAP70). In addition, in the case of the experimental group treated with hIgG4, the amount of phosphorylated ZAP70 was smaller than that of the positive control group. In contrast, in the case of the experimental group treated with the anti-CEACAM1 antibody, the amount of the phosphorylated ZAP70 measured was similar to that of the positive control group.

### Example 7.2. Confirmation of Increase in Expression Level of NFAT Transcription Factor

In order to prepare Jurkat cells expressing NFAT and CEACAM1, Jurkat cells expressing CEACAM1, prepared in Example 7.1, with a cell number of 3×10⁶ were transfected with 10 μg of pGL4.30 [luc2p/NFAT-RE/Hygro] and 1 μg of pTurbo RFP-C plasmid using a Neon transfection system (1,400 voltage/20 ms/2 pulse). After 72 hours, the transfected Jurkat cells were harvested, and the Jurkat cells expressing RFP were sorted using a flow cytometer (FACSAria). The sorted Jurkat cells were cultured in a culture medium containing 1 mg/mL of G418 (Sigma, Cat. No. G8168) and 0.5 mg/mL of hygromycin B (Invitrogen, Cat No. 10687010). In particular, the same culture medium used in Example 7.1 was used.

Jurkat cells overexpressing NFAT and CEACAM1 were dispensed with a cell number of 3×10⁶ cells per well, treated with hIgG4 (Sigma, Cat. No. 14639) or anti-CEACAM1 antibody, and then stimulated with 0.05 μg/mL of anti-CD3 antibody (eBioscience, Cat.No. 16-0037-85). In particular, the hIgG4 (Sigma, Cat. No. 14639) or anti-CEACAM1 antibody was serially diluted 3-fold starting from a concentration of 30 μg/mL and used. After 24 hours, 100 μL of the supernatant was transferred to a 96-well well plate, and treated with 100 μL of Bright-Glo luciferase assay system (Promega, Cat. No. E2610). Thereafter, the fluorescence was measured using a GloMax Discover multimode microplate reader.

In particular, the Jurkat cells overexpressing NFAT and CEACAM1, which were stimulated with the anti-CD3 antibody and treated with hIgG4, were set as a control group, and the Jurkat cells overexpressing NFAT and CEACAM1, which were treated with the anti-CEACAM1 antibody, were set as an experimental group.

As a result, as shown in FIG. 14, it was confirmed that the fluorescence intensity was increased in a concentration-dependent manner in the experimental group, and from this result, that the expression level of NFAT was increased.

### Example 7.3. Confirmation of Increase in IL-2 Expression Level

First, a 96-well-plate was coated with 1 μg/mL of the anti-CD3 antibody (OKT-3) at 4° C. overnight. Each well was washed twice with cold DPBS before adding the cells. Then, Jurkat cells overexpressing CEACAM1 prepared in Example 7.1 were dispensed with a cell number of 1×10⁵ into each well, 200 μL each of the anti-CEACAM1 CEACAM1 antibody was added at a concentration of 10 μg/mL thereto and cultured for 3 days. Thereafter, the supernatant was collected, and the expression level of IL-2 was measured using an IL-2 ELISA kit (BD Bioscience, Cat No. 550611).

In particular, the Jurkat cells overexpressing CEACAM1, which were stimulated with the anti-CD3 antibody and treated with hIgG4 were set as a control group, and Jurkat cells overexpressing CEACAM1, which were treated with an anti-CEACAM1 antibody, were set as an experimental group.

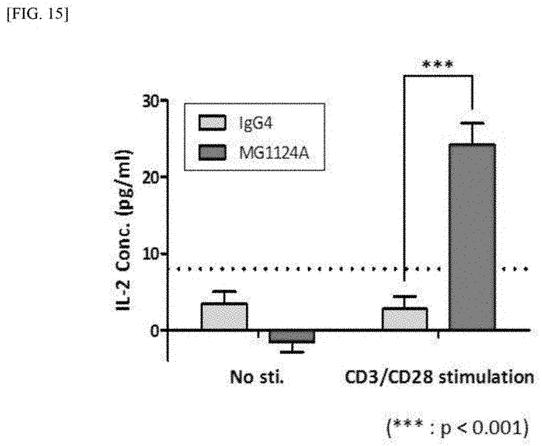

As a result, as shown in FIG. 15, it was confirmed that the IL-2 expression level in the experimental group was significantly increased compared to the control group.

1. A conformational epitope consisting of 4 to 69 amino acid residues of the sequence of amino acids corresponding to positions 35 to 141 of carcinoembryonic antigen-related cell adhesion molecule 1 (CEACAM1), wherein the conformational epitope comprises any one amino acid selected from the group consisting of the amino acids at positions 63, 64, 66, 68, 75, 76, 78, 83, 86, 90, 123, 125, 129, and 131, and a combination thereof, and wherein the amino acid positions are according to SEQ ID NO: 1.

2. The conformational epitope of claim 1, wherein the CEACAM1 consists of the amino acid sequence of SEQ ID NO: 1.

3. The conformational epitope of claim 1, wherein the 63rd amino acid is phenylalanine (Phe), the 64th amino acid is glycine (Gly), the 66th amino acid is serine (Ser), the 68th amino acid is tyrosine (Tyr), the 75th amino acid is glycine (Gly), the 76th amino acid is asparagine (Asn), the 78th amino acid is glutamine (Gln), the 83rd amino acid is alanine (Ala), the 86th amino acid is threonine (Thr), the 90th amino acid is threonine (Thr), the 123rd amino acid is glutamine (Gln), the 125th amino acid is isoleucine (Ile), the 129th amino acid is leucine (Leu), and the 131st amino acid is asparagine (Asn).

4. The conformational epitope of claim 1, wherein the conformational epitope comprises amino acids at amino acid positions 63, 64, 66, 68, 75, 76, 78, 83, 86, 90, 123, 125, 129, and 131 of CEACAM1.

5. An anti-carcinoembryonic antigen-related cell adhesion molecule 1 (CEACAM1 antibody or a fragment thereof, which specifically binds to a conformational epitope consisting of 4 to 69 amino acid residues of CEACAM1 between position 35 and position 141 with reference to SEQ ID NO: 1, wherein the conformational epitope comprises any one amino acid selected from the group consisting of the amino acids at positions 63, 64, 66, 68, 75, 76, 78, 83, 86, 90, 123, 125, 129, and 131, and a combination thereof.

6. The anti-CEACAM1 antibody or fragment thereof of claim 5, which specifically binds to a conformational epitope consisting of 4 to 69 amino acid residues of CEACAM1 between position 35 and position 141 with reference to SEQ ID NO: 1 wherein the conformational epitope comprises the amino acids at positions 63, 64, 66, 68, 75, 76, 78, 83, 86, 90, 123, 125, 129, and 131.

7. The anti-CEACAM1 antibody or fragment thereof of claim 5, wherein the antibody or fragment thereof binds to CEACAM1 within an intermolecular distance of 4.5 Å.

8. The anti-CEACAM1 antibody or fragment thereof of claim 5, wherein the antibody or fragment thereof binds to CEACAM1 by van der Waals, hydrophobic binding, or electrostatic binding.

9. The anti-CEACAM1 antibody or fragment thereof of claim 5, wherein the antibody or fragment thereof has a binding affinity for CEACAM1 of less than 1×10⁻⁸ KD (M).

10. A polynucleotide encoding the antibody or fragment thereof of claim 5.

11. An expression vector comprising the polynucleotide of claim 10.

12. A transformed cell into which the expression vector of claim 11 is introduced.

13. A method for producing an antibody or a fragment thereof comprising culturing the transformed cell of claim 12.

14. A composition comprising the antibody or fragment thereof according to claim 5 as an active ingredient.

15. (canceled)

16. (canceled)

17. A method for preventing or treating cancer in a subject in need thereof, comprising administering an effective amount of the antibody or fragment thereof of claim 5 to the subject.

18. The anti-CEACAM1 antibody or fragment thereof of claim 5, wherein the antibody or fragment thereof comprises: a heavy chain CDR1 including the amino acid sequence represented by SEQ ID NO: 2, a heavy chain CDR2 including the amino acid sequence represented by SEQ ID NO: 3, a heavy chain CDR3 including the amino acid sequence represented by SEQ ID NO: 4, a light chain CDR1 including the amino acid sequence represented by SEQ ID NO: 5, a light chain CDR2 including the amino acid sequence represented by SEQ ID NO: 6, and a light chain CDR3 including the amino acid sequence represented by SEQ ID NO: 7.

19. The anti-CEACAM1 antibody or fragment thereof of claim 5, wherein the antibody or fragment thereof comprises a heavy chain variable region comprising the amino acid sequence of SEQ ID NO: 8; and a light chain variable region comprising the amino acid sequence of SEQ ID NO: 9.

20. The anti-CEACAM1 antibody or fragment thereof of claim 5, wherein the antibody fragment is selected from a Fab, a Fab′, a F(ab′)2, a scFv, minibody, or a diabody.
